(19) United States
(12) Patent Application Publication
(10) Pub. No.: US 2008/0243247 A1
(43) Pub. Date: Oct. 2, 2008

(54) METHOD AND APPARATUS FOR PREVENTION AND TREATMENT OF ADULT GLAUCOMA

(76) Inventors: Brooks J. Poley, Bluffton, SC (US); Richard L. Lindstrom, Wayzata, MN (US)

Correspondence Address: WESTMAN CHAMPLIN & KELLY, P.A., SUITE 1400, 900 SECOND AVENUE SOUTH, MINNEAPOLIS, MN, 55402-3244, US

(21) Appl. No.: 12/108,712

(22) Filed: Apr. 24, 2008

Related U.S. Application Data
(63) Continuation-in-part of application No. 11/728,381, filed on Mar. 26, 2007.
(60) Provisional application No. 60/926,568, filed on Apr. 27, 2007.

(51) Int. Cl. A61F 2/16

(52) U.S. Cl. 623/6.27; 623/6.43

(57) ABSTRACT

An apparatus and method for reducing intraocular pressure in a patient's eye is provided. A deformable intraocular member having a relaxed state with a relaxed profile having a first width, a first energized state with a first profile having a second width, and a second energized state with a second profile having a third width is provided. The deformable intraocular member relaxed state width is greater than the width of a lens capsule which surrounds a lens of the patient's eye prior to cataract surgery. The second energized state width is greater than the patient's lens capsule width after cataract surgery and smaller than the width in the relaxed state. The deformable intraocular member in the second energized state is configured to urge deformation of the lens capsule to thereby promote opening of a trabecular meshwork and canal of Schlemm of the patient's eye to promote drainage of aqueous humor from an anterior chamber and reduce intraocular pressure in the patient's eye.

### CROSS-REFERENCE TO RELATED APPLICATION

The present application is based on and claims the benefit of U.S. provisional patent application Ser. No. 60/926,568, filed Apr. 27, 2007; and is also a Continuation-In-Part of patent application Ser. No. 11/728,381, filed Mar. 26, 2007, the contents of which are hereby incorporated by reference in their entirety.

### FIELD OF THE INVENTION

The present invention relates to the human eye. More specifically, the present invention relates to glaucoma of the eye.

### BACKGROUND OF THE INVENTION

The lens of the human eye is suspended in the eye by ligaments called zonule fibers. The lens is impressed against the iris. The eye has three chambers. The anterior chamber is roughly defined between the iris and the cornea. The posterior chamber is between the iris, zonule fibers and the lens. The vitreous chamber is between the lens and the retina. The anterior chamber and the posterior chamber are filled with aqueous humor. The vitreous chamber is filled with a more viscous fluid, the vitreous humor.

As a person ages, each year the lens becomes less compressible. A ten-year-old child's lens is so malleable it is able to easily change its shape and adjust its focus from six inches to infinity. By age 55 years the lens is so rigid it can no longer change its shape. It has lost all its accommodation. It can only focus at one distance. In addition the aging lens enlarges and moves forward in the eye.

Further, the anterior chamber of the eye shallows and the intraocular pressure razes with increasing age. Shallowing of the anterior chamber results from forward movement of the iris diaphragm. The aging lens as it enlarges and moves forward within the eye causes the forward iris movement and shallowing of the anterior chamber. Ninety-eight percent of 10 year olds have flat iris diaphragms, and deep anterior chambers. By 80 years of age less than 5% of eyes retain flat iris diaphragms and deep anterior chambers.

Liquid (aqueous humor) in the anterior chamber normally drains through the Canal of Schlemm first passing through the trabecular meshwork. Any factor that compromises the ability of fluid to drain through the trabecular meshwork and through the Canal of Schlemm can cause a pressure elevation in the anterior chamber which leaves the eye vulnerable to damage.

However, the functioning of the filtering trabecular meshwork decreases with age. As the eye ages, increased intraocular pressure is necessary to force aqueous through this failing filtering tissue in its passage from the eye. When the pressure rises above normal, glaucoma occurs. At age 20 less than 0.5% of patients have glaucomatous intraocular pressures. By age 80 years over 4% of patients have glaucomatous pressures.

The most common forms of glaucoma develop when these drainage passages become clogged over time. The inner eye pressure (also called intraocular pressure or IOP) rises because the correct amount of fluid can't drain out of the eye. The increase pressure results in damage to the optic nerve.

Three primary types of adult glaucoma are Narrow Angle Glaucoma, Chronic Narrow Angle Glaucoma and Open Angle Glaucoma. Current conventional thinking focuses on the trabecular meshwork as the problem in these types of glaucoma.

In Narrow Angle Glaucoma, the forward position of the iris blocks aqueous from the trabecular meshwork. A dramatic increase in pressure causes an acute glaucoma crisis.

In Chronic Narrow Angle Glaucoma, the space between the iris and cornea is narrowed, but the narrow space still allows aqueous to flow to the trabecular meshwork. Current therapy employs drops, pills, or laser to decrease aqueous production, or make the compromised trabecular meshwork and other outflow channels more efficient.

In Open Angle Glaucoma aqueous passage from the anterior chamber to the trabecular meshwork is without obstruction. This glaucoma is treated the same as Chronic Narrow Angle Glaucoma. The same therapy is used to decrease the aqueous production, or increase the function of the trabeculum and other out flow channels.

In all three glaucomas damaged tissue is treated, the iris in the case of Narrow Angle Glaucoma, or the trabeculum in the case of Chronic Narrow Angle Glaucoma or Open Angle Glaucoma.

### SUMMARY OF THE INVENTION

An apparatus and method for reducing intraocular pressure in a patient's eye is provided. A deformable intraocular member having a relaxed state with a relaxed profile having a first width, a first energized state with a first profile having a second width, and a second energized state with a second profile having a third width is provided. The deformable intraocular member relaxed state width is greater than the width of a lens capsule which surrounds a lens of the patient's eye prior to cataract surgery. The second energized state width is greater than the patient's lens capsule width prior to cataract surgery and smaller than the width in the relaxed state. The deformable intraocular member in the second energized state is configured to urge deformation of the lens capsule to thereby promote opening of a trabecular meshwork and canal of Schlemm of the patient's eye to promote drainage of aqueous humor from an anterior chamber and reduce intraocular pressure in the patient's eye.

### BRIEF DESCRIPTION OF THE DRAWINGS

FIG. 1 is a sagittal horizontal sectional drawing of an adult human eye (prior art).

FIG. 2 schematically depicts the stages of a human eye from a normal eye, to an aging eye, to an eye treated for glaucoma.

FIG. 3 is a simplified block diagram showing steps in accordance with counseling a patient.

FIG. 4 is a simplified block diagram showing steps in accordance with exchanging a natural lens of a patient with an artificial one.

FIG. 5 is a simplified block diagram showing steps of the present invention including conducting an eye exam, counseling a patient and performing a lens exchange.

FIG. 6A is a cross-sectional view of a patient's eye including a lower portion with a natural lens and an upper portion with an intraocular insert with accordance with one example embodiment.

FIG. 6B is a cross-sectional view of a patient's eye including a lower portion with a natural lens and an upper portion with an intraocular insert with accordance with one example embodiment.

FIG. 6C is a cross-sectional view of a patient's eye including a lower portion with a natural lens and an upper portion with an intraocular insert with accordance with one example embodiment.

FIG. 7A is a plan view of an artificial intraocular lens including haptic.

FIG. 7B is a side plan view of the lens of FIG. 7A.

FIG. 7C is a plan view of the lens of FIG. 7A following implantation into a capsular sac.

FIG. 8A is a plan view of a capsular tension ring in a relaxed state.

FIG. 8B is a plan view of the capsular tension ring of FIG. 5A following implantation to a capsular sac of a patient's eye.

FIG. 9A is a side cross-sectional view of a lens in a capsular sac.

FIG. 9B is a side cross-sectional view of the lens of FIG. 9A in the capsular sac which further includes an implanted capsular tension ring.

FIG. 10A is a cross-sectional view showing a configuration of a prior art haptic.

FIG. 10B is a cross-sectional view of a haptic in accordance with one example embodiment of the present invention.

FIG. 11A is a plan view of a capsular tension ring.

FIG. 11B shows the capsular tension ring during the implantation process in a energized state.

FIG. 11C shows the capsular tension ring following the implantation process.

FIG. 11D shows the capsular tension ring and an intraocular lens following implantation in a patient's eye.

### DETAILED DESCRIPTION OF ILLUSTRATIVE EMBODIMENTS

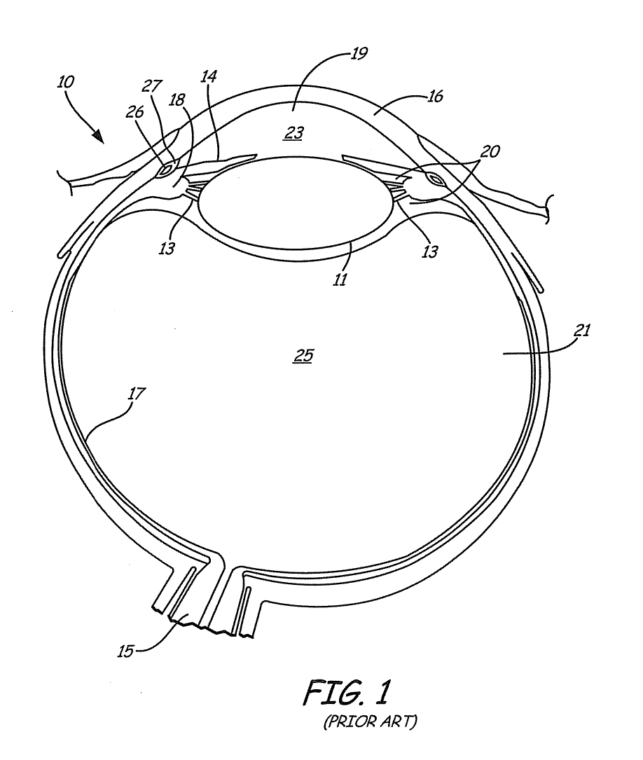

FIG. 1 illustrates an adult human eye indicated generally at 10. Eye 10 has a lens 11 suspended in the eye by zonule fibers 13. The lens is located just behind the iris 14. The purpose of the lens is, to focus light onto the retina 12 which is a multi-layered sensory tissue that lines the back of the eye. The optic nerve 15 transmits electrical impulses from the retina to the brain. The cornea 16 is the transparent dome shaped window covering the front of the eye. A cornea incision 17 is also shown through which surgery may be performed as discussed below.

The anterior chamber 19 is the space between the iris 14 and the cornea 16. It is filled with aqueous humor 23 which is a thin watery fluid continuously produced by the ciliary body 18, the part of the eye that lies just behind the iris 14.

The canal of Schlemm 26 lies at the root of the iris 14, in front of the ciliary body 18, inside the cornea 16. The trabecular meshwork 27 separates the canal of Schlemm 26 from the anterior chamber space 19. Aqueous humor 23 flows through the trabecular mesh work 27, into the canal of Schlemm 26, to collector channels and then to aqueous veins outside the eye.

The posterior chamber 20 is the space between the iris 14, zonular fibers 13 and the lens 11. It is filled with aqueous humor. The vitreous chamber 21 is the space between the lens 11 and the retina 12. It is filled with the vitreous humor 25, a viscous-like liquid.

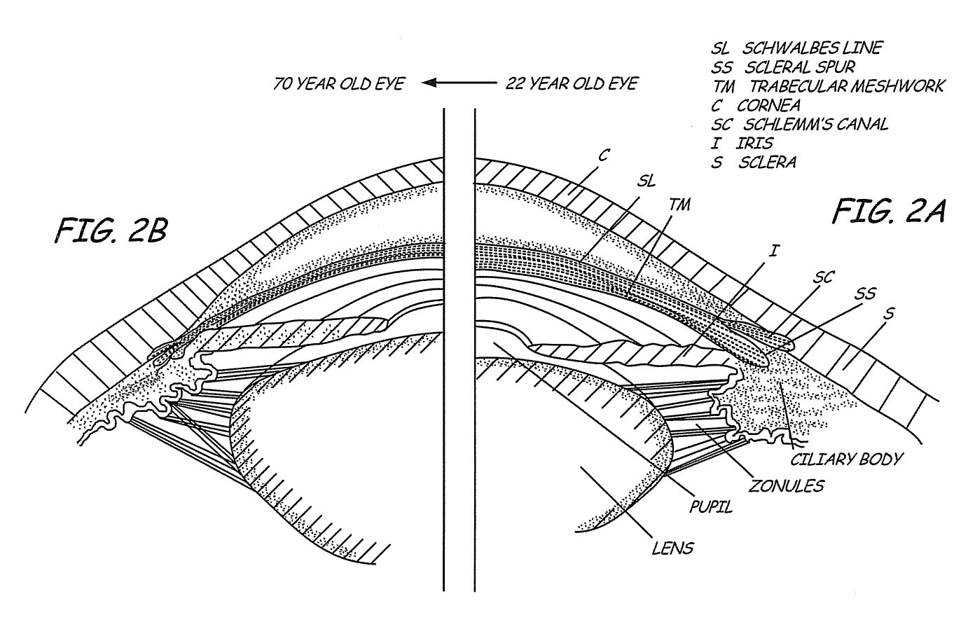

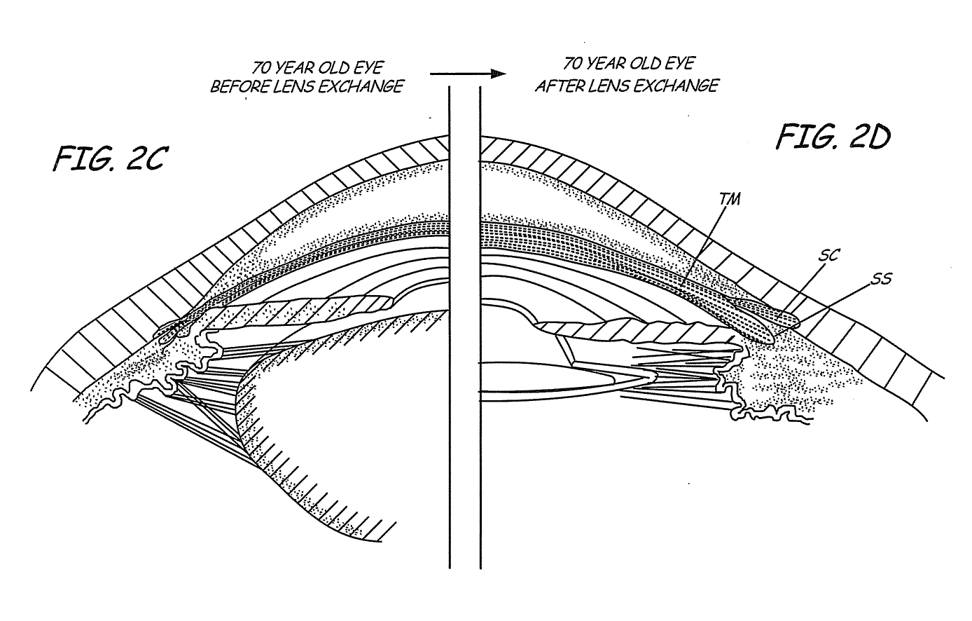

As the lens ages, it enlarges and moves forward within the eye. The iris diaphragm is forced forward toward the cornea, and the anterior chamber shallows (FIG. 2B). These lens changes also affect the zonules, the morphology of the ciliary body, their muscles, and the trabecular meshwork.

The enlarging and forward moving lens causes the trabecular meshwork function to becomes less efficient. Aqueous fluid is less able to transition through this filtration meshwork. The aging lens alters the position of the zonules, the morphology of the ciliary body, and its muscles. The changed orientation of the zonules, ciliary body, and its muscles influence the trabecular meshwork. It has been shown that tendons of the longitudinal ciliary body muscles traverse the entire trabecular meshwork. They are an integral part of the plates of the trabecular meshwork. As the tension on the tendons change, the alignment of the trabecular plates is altered. By altering the position of the trabecular plates, the spaces between the plates are diminished. Aqueous fluid has less space through which to pass. This causes the intraocular pressure to rise to force aqueous through these diminished spaces. In this way the trabecular meshwork becomes a less efficient filtering bed, intraocular pressure rises, and glaucoma occurs.

The aging lens is a cause of adult glaucoma. The method of treating existing glaucoma as well as a treatment for the prevention of glaucoma includes first counseling the patient about glaucoma and the treatment of glaucoma by lens replacement. This is followed by removal of the offending source of the glaucoma, namely the enlarging or enlarged lens, through a lens replacement procedure. In such a procedure, the natural lens is removed and replaced with an artificial one.

More specifically the invention contemplates counseling the patient on the causes of glaucoma. This can include showing the patient visual aids such as a diagram of the eye which can be a diagram of a healthy eye or a diagram of a diseased eye; counseling the patient on how glaucoma damages the eye; explaining lens exchange to the patient and how it corrects glaucoma when treated early, or makes glaucoma control easier when treated later; counseling the patient on how a lens exchange procedure will improve vision and restore accommodation; and explaining the patients experience upon the choice of the lens replacement procedure. The invention then contemplates surgical intervention through the performance of a lens transplant procedure which will usually be phacoemulsification and implantation of artificial lens.

The lens, as it ages within the eye, becomes the primary cause of the glaucomas discussed. Its continued presence creates secondary changes within the anterior segment of the eye causing elevated eye pressure, and subsequent eye damage namely loss of retinal visual cells and atrophy of retinal neurons best identified as cupping of the optic nerve head.

Focusing on the lens as the cause of these major glaucomas and performing a lens exchange removes the offending or potentially offending lens and replaces it with an artificial lens. The anterior segment of the eye is allowed to revert to a normal, stable anatomic state. Since the lens is removed, it can no longer compromise adjacent tissues which when damaged cause glaucoma. This is shown in FIG. 2C which shows a typical 70 year old eye after lens exchange.

The eyes of the three glaucomas here discussed all possess an aging and enlarging lens, which forces the iris forward, and compromises the trabecular meshwork. In Acute Glaucoma the iris touches the cornea. In Chronic Narrow Angle Glaucoma the iris is far forward, but not touching the cornea. In Open Angle Glaucoma the iris is 30% forward of where it was at age 22 years. Incorrectly, this iris location is considered normal.

### Narrow Angle Glaucoma

Narrow Angle Glaucoma occurs when the peripheral iris contacts the inner corneal wall. The normal passage of aqueous is blocked in its movement from the anterior chamber to the trabecular meshwork on its way to the canal of Schlemm. When this occurs, the abrupt pressure rise creates an acute glaucoma attack.

### Conventional Treatment

Conventional treatment creates a hole in the peripheral iris. (An iridectomy by surgery or an iridotomy by laser). This maneuver dramatically reduces the elevated pressure. It creates an alternative passage for aqueous produced by the ciliary body behind the iris to move through the peripheral iris hole directly into the trabecular meshwork, on to the canal of Schlemm, and out of the eye. The space between the iris and corneal wall is deepened where the hole is created. The depth of the anterior chamber is deepened mostly near the hole's location, but less deepened throughout the remainder of the circumference of the peripheral iris. However, this maneuver does not reestablish the normal depth of the anterior chamber. Nor does it allow the iris to reestablish its more normal flatter position within the anterior segment of the eye.

Although the acute attack of glaucoma has been relieved, the cause of the forward iris position has not been corrected. The primary cause of the glaucoma attack is the forward positioned surface of the now abnormal lens. Over time, as this lens continues to enlarge and move forward, one adverse event can occur, and another adverse event will occur. If the created hole is small, it may again block as the iris continues to advance forward, and a second acute attack can occur. The once controlled Acute Narrow Angle glaucoma can over time convert to Chronic Narrow Angle Glaucoma. When this occurs, conventional treatment will advocate the use of eye drops, laser, or when the condition deteriorates enough, a conventional filtering operation.

### Lens Exchange for Narrow Angle Glaucoma

The acute pressure elevation should be managed medically in a timely manner. Once the pressure is lowered, the use of lens exchange for this glaucoma will return the iris to its normal position, and the anterior chamber will regain its normal depth. The normal passage of aqueous will be restored. Aqueous will again move from behind the iris, through the pupil, to the periphery of the anterior chamber, into the trabecular meshwork, and canal of Schlemm, and on out of the eye.

Not only will the normal relationships of the anterior chamber be reestablished, but also these normal relationships will be permanent. These normal relationships will persist because the offending agent, the enlarged lens, will be gone. Neither a second attack of Narrow Angle Glaucoma can occur, nor can a Chronic Narrow Angle Glaucoma develop over time.

### Chronic Narrow Angle Glaucoma

Conventional knowledge describes Chronic Narrow Angle glaucoma as a condition where the eye's pressure is elevated, the anterior chamber is very shallow, and the peripheral iris encroaches on anterior chamber angle, but does not block it.

### Conventional Treatment

Conventional treatment is aimed at compensating for the reduced ability of the trabecular meshwork and collecting aqueous veins to transport aqueous from the eye. Pills, eye drops, and lasers are used to reduce the production of aqueous from the ciliary body. Other eye drops and lasers are used to increase the aqueous outflow of the trabecular meshwork and collecting aqueous veins. By reducing the production, or increasing the outflow of aqueous, the eye's pressure is lowered.

When these treatments no longer control the pressure, a filtering operation is performed. Here, the normal outflow channels are abandoned: they are bypassed. A hole is created through the shell of the eye at the corneal/scleral junction. A trabeculectomy is performed. The aqueous now passes from the anterior chamber through the corneal/scleral hole, and under tenon's capsule outside the shell of the eye where it is returned to the orbital vascular circulation.

### Lens Exchange for Chronic Narrow Angle Glaucoma

The forward iris position identifying this type of glaucoma is anatomically accurate, but this forward iris position is not the cause of the failed trabecular function.

Again, the lens is causing the glaucoma. The aging lens not only grows from front to back, but also from side to side. This lens enlargement in all directions compromises the adjacent structures of the eye's anterior segment. The aging lens alters the morphology of the ciliary body and its muscles. Their altered position alters the tension of the ciliary body muscle tendons traversing the trabecular meshwork. The trabecular plates of the meshwork are reoriented diminishing the spaces between the plates. The diminished space through which the aqueous must pass creates more resistance for aqueous to flow from the eye and glaucoma results.

By removing the aging lens and implanting an artificial lens, the compromising force of the offending lens is removed. The structures compromising the trabecular meshwork revert to their previous normal positions within the anterior segment. The abnormal tension transmitted by the ciliary body muscle tendons to the trabecular plates is relieved. The trabecular plates are stabilized, and the trabecular spaces thereafter remain open. The cascading events causing the increasing resistance to aqueous outflow are permanently eliminated. The slowly increasing pressure of the eye will be halted. The eye's pressure will lower during the next few months, and remain stable thereafter.

### Chronic Open Angle Glaucoma

This condition is identified as an eye with elevated pressure whose anterior chamber is deep, and the trabeculum has open access to aqueous passing from the anterior chamber.

### Conventional Treatment

Conventional treatment is the same as for previously described Chronic Narrow Angle Glaucoma. Eye drops, pills, and lasers are used to lower the aqueous production, or increase aqueous outflow from the eye. Again, when these no longer work, a trabeculectomy operation is performed.

### Lens Exchange for Chronic Open Angle Glaucoma

It is conventionally incorrectly assumed the anterior chamber and iris position is normal in a patient with Chronic Open Angle Glaucoma. These glaucoma patients' anterior chambers have all shallowed 30% or more over time. The usual enlargement of the lens as it ages causes forward movement of the iris which shallows the anterior chamber depth in all people all the time. Lens exchange for this condition has the previously mentioned advantages.

The treatment for Chronic Narrow Angle, and Chronic Open Angle Glaucoma, and Open Angle Glaucoma is the same. The aged enlarged lens is removed and replaced with an artificial lens.

The anterior chamber depth, iris position and ciliary body revert to locations similar to where they were when the patient was 22 years of age. The anterior segment is now stabilized. This stabilization allows the trabecular meshwork to function normally, and the pressure to be controlled.

If the lens exchange is done early in the detection of the glaucoma, the pressure will revert to normal and should remain normal thereafter. If glaucoma has existed for a great deal of time, lens exchange will still be of benefit. In some cases the pressure will still revert to normal and remain normal. In other cases, all trabecular damage will not be reversed. In these cases, medical treatment is indicated to maintain adequate control. In almost no cases will the pressure control require more medical treatment than prior to the lens exchange.

### Counseling Lens Exchange to Treat Glaucoma

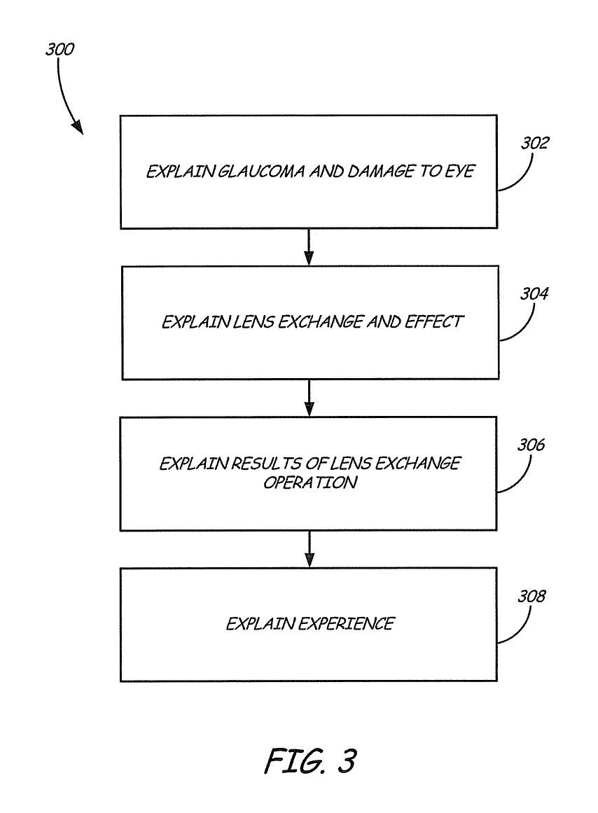

FIG. 3 is a block diagram 300 of an example of providing counseling to a patient as a part of the method of treating glaucoma and/or treating a pre-glaucoma condition.

### 1. Explain Glaucoma and how it Damages the Eye (302).

With the use of visual aids as may be appropriate, counsel how aqueous fluid is made within the eye. When it encounters increased resistance in its passage through the eyes out flow channel, the eye's pressure elevates, and glaucoma occurs. The eye's out-flow channel is called the trabecular meshwork. If the eye's trabecular meshwork is compromised, pressure elevates above normal, and retinal visual cells and the optic nerve are damaged. Damage to these structures causes visual loss. If this damage continues long enough, useful vision is lost.

2. Explain Lens Exchange, how it can Stabilize Glaucoma Suspect and Keep them from Developing Glaucoma, how it Corrects Glaucoma when Treated Early, or Makes Glaucoma Control Easier when Treated Later (304).

With the use of visual aids counsel how the lens exchange operation is a new alternative prevention and treatment for glaucoma. This treatment recognizes failure of the eye's outflow channel, the trabecular meshwork, is caused by the enlarging aging lens within the eye. By replacing the aging lens with a stable artificial lens, the cause of the trabecular failure is removed. Thereafter, the eye's filtering outflow channel is allowed to resume its normal function, and glaucoma is controlled.

If lens exchange is accomplished when glaucoma is first detected or early in the disease, the trabecular meshwork can recover its normal function, and normal eye pressures can resume without further treatment.

Lens exchange is still indicated when accomplished at a later stage in the disease. Later in the disease, some trabecular damage may have occurred. The out flow channel will nevertheless be stabilized by the operation. Some times the trabecular meshwork will still retain enough filtering ability to allow the eye pressure to stabilize at a normal rate without need of further treatment. If the trabecular meshwork is more damaged, it will be stabilized at this level. Eye drops may be necessary to normalize the pressure. In either case, early or late, lens exchange will benefit patients with glaucoma.

### 3. Explain how the Operation Will Improve Vision, and Restore Focusing Ability (Accommodation) (306).

Although lens exchange is here performed to correct the glaucoma, by replacing the patient's aging lens with an artificial lens, the usual visual benefits of the Lens Exchange will still occur. The eye's optical imperfections previously causing the need for glasses will be corrected. The surgeon will select the correct power of artificial lens to implant during the Lens Exchange. If the patient does not have astigmatism, glasses should no longer be needed. If the surgeon selects a multifocal artificial lens, the patient's accommodation will also be corrected, and reading glasses should no longer be needed.

4. Explain the Patient's Experience if they Chose this New Glaucoma Procedure. (308)

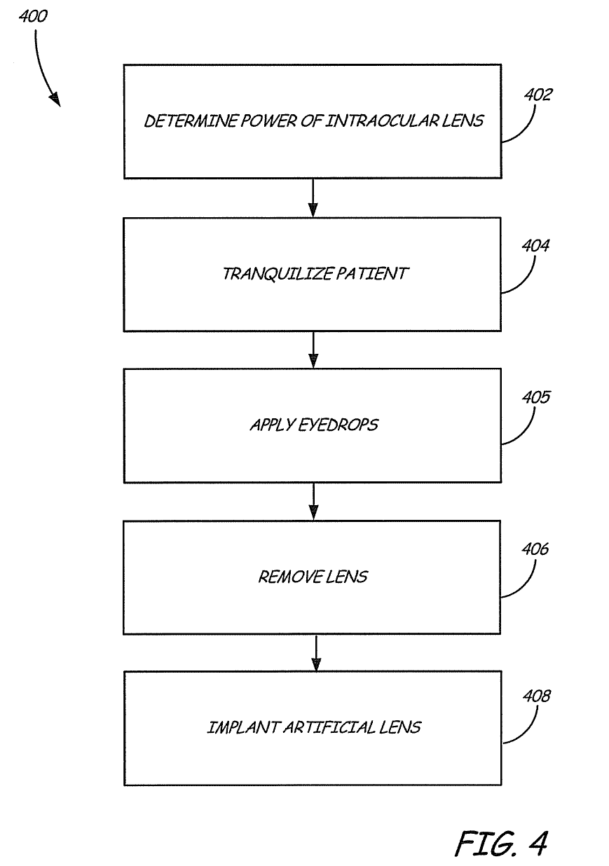

FIG. 4 is a block diagram 400 showing steps of the invention. Prior to surgery, the surgeon's staff will calculate the power for the intraocular lens at step 402. Surgery is an outpatient procedure. An anesthesiologist will start an IV so tranquilizing medicine can be given during the operation 404. Eye drops put the eye to sleep 405. The lens is removed 406 utilizing the regular phacoemulsification operation, and the artificial lens implanted 408. This operation usually takes from ten to twenty minutes. The incision is small enough that only a single suture or no suture is necessary for wound closure.

Typically, the patient can be up and about at home, but resting the day of surgery. The day following surgery, s/he is seen in the surgeon's office, and the eye checked for pressure, vision, and healing. Eye checks are usually done one day, one week, three weeks and six weeks after surgery.

Full activity can be resumed two or three days following surgery. An eye shield is recommended for several weeks for sleep. Eye patches are usually unnecessary after the first day.

The above steps can be accomplished with the use of visual aids as may be appropriate to enable a full understanding and appreciation of the treatment by the patient.

Glaucoma patients treated with lens exchange who experience normal eye pressures should consider their status shifted from one having glaucoma to being a glaucoma suspect. Although their pressures are normalized, patients should be checked periodically to assure the pressures remain correct.

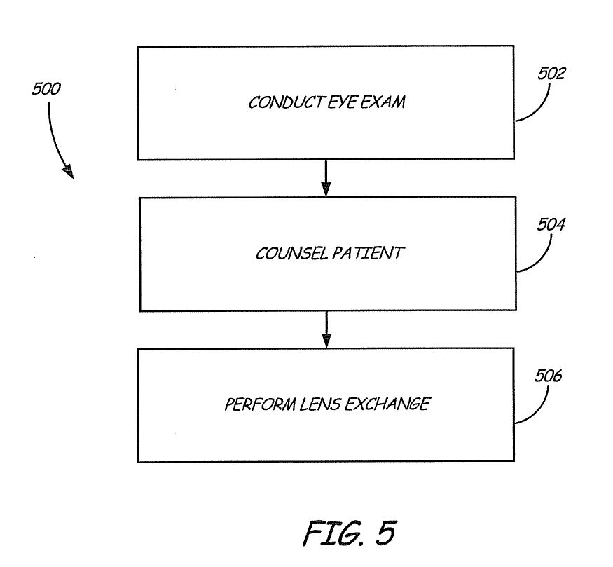

FIG. 5 is a simplified block diagram 500 showing general steps in accordance with the present invention. At block 502, an eye examination is conducted on the patient. If it appears that the patient has glaucoma, or may have glaucoma in the future, at block 504, the patient can be counseled using the techniques discussed above. Based upon the decision of the patient through the step of counseling at block 504, a lens exchange is performed at block 506. The block diagram at FIG. 5 is greatly simplified and each of the individual blocks illustrated at 502, 504 and 506 may contain numerous additional steps.

In one aspect, the present invention includes intraocular inserts including configurations of lenses and capsular tension rings, and methods, for use in connection with the above described procedures. For example, in one configuration, a lens is provided which is utilized in combination with cataract surgery or refractive surgery to enhance the lowering of the intraocular pressure. Further, the configuration can lower the pressure for ocular hypertensive in glaucoma patients. As used herein, a hypertensive eye has an intraocular pressure which is 20 mm Hg. Or greater.

In one configuration, a lens having a posterior vault is provided. The vaulting can be of any appropriate amount or configuration. In one configuration, the vaulting is between about 1° and about 20°. In another configuration, the vaulting is between about 3° and about 10°. With such an intraocular lens configuration, an A-constant of greater than about 118.0 is provided. In another configuration, the A-constant is greater than about 118.5.

In another configuration, a multi-piece lens is provided having non-angled haptics is provided. In another configuration, a plate lens having non-angled, or flat, haptics is provided. In such a configuration, the A-constant can be greater than about 118.0. In a further configuration, the A-constant of such a lens is greater than 118.5.

The lenses can be made or fabricated of any appropriate material or combination of materials. Example materials include PMMA (Polymethyl methacrylate), silicone or acrylic. A single piece or multi-piece lenses can be utilized. In one configuration, a plate haptic is provided or a loop.

In general these lens configurations of the present invention are provided or configured to open the trabecular mesh work thereby allowing a reduction in the intraocular pressure. In one configuration, the intraocular lens is set back in the eye whereby the zonules are pulled or otherwise drawn back to thereby open the trabecular mesh work.

In another example configuration, a capsular tension ring is provided to reduce the intraocular eye pressure. The capsular tension ring can be any appropriate ring configuration such as those typically used to repair a damaged capsular bag.

Further, the present invention includes a method of combining cataract surgery with the implantation of an intraocular lens, and/or a capsular tension ring, to lower pressure in an ocular hypertensive patient, or in a glaucoma patient. The particular lens configuration can be those discussed above. For example, a vaulted lens, a lens with an A-constant of 118 or greater, or 118.5 or greater, or a flat lens, which is either a multi-piece lens with non-angular haptics or a plate lens with non-angled haptics.

The vaulting of the vaulted lens can be configured as desired. In one configuration, the vaulting occurs after implantation under the influence of the capsular bag.

When implementing a capsular tension ring, the ring can be configured to “stretch” to capsular bag thereby applying tension to the zonules and effecting increased facility of outflow through the trabecular meshwork. The diameters of the intraocular lens and the capsular tension ring are preferably greater than about 12 mm having a size prior to compression which is greater than the contracting capsular bag, typically about 10 to 11 mm in diameter. In one configuration, the size is between 12 and about 14 mm. In another configuration, the size is between about 12 and about 20 mm.

In the various configurations of the present invention, stretching is applied to the capsular bag to thereby increase outflow of aqueous humor fluid from the anterior chamber. In another configuration, posterior vaulting is provided. In yet a further configuration, the intraocular lens is positioned substantially posterior.

With the present invention, a method and apparatus are provided in which an intraocular lens capsule insert is used to reshape the lens capsule of a patient's eye and is configured to provide a shape and/or arrangement to the lens capsule which urges the lens capsule to change shape and/or position in a manner which promotes opening of the trabecular meshwork of the patient's eye. This allows aqueous humor to drain from the patient's eye and reduce intraocular pressure in the eye. This can be achieved using any appropriate intraocular insert including an intraocular lens, capsular tension ring, or other type of intraocular insert.

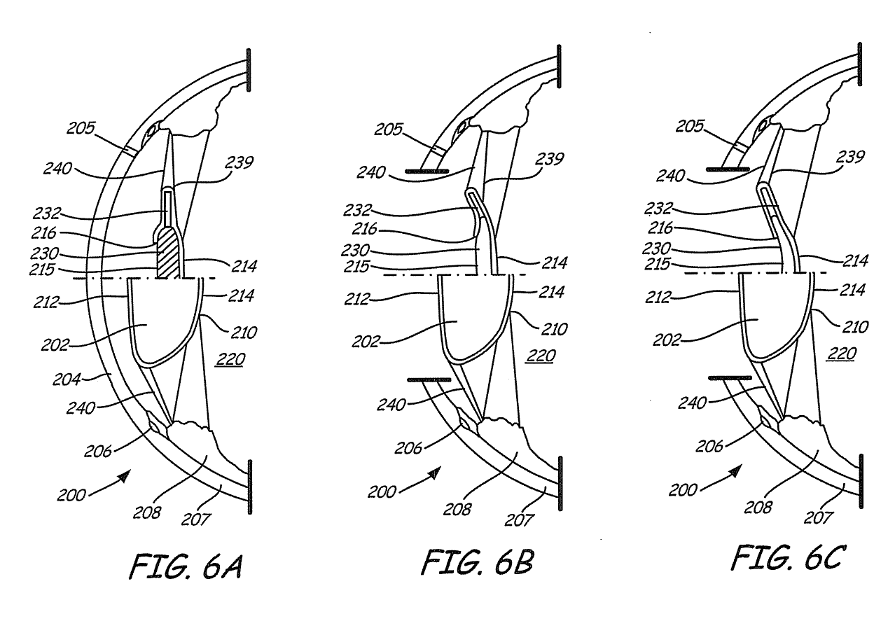

FIGS. 6A, 6B and 6C are split cross-sectional views of a patient's eye 200 showing various example configurations of the present invention and examples of the reshaping of the lens capsule 210. As illustrated in FIG. 6A-C, the lower portion of the Figures show the eye 200 with a natural lens 202. Eye 200 includes a cornea 204, a canal of schlemm/trabecular meshwork 206 and capillary body muscles 208 which attach to lens capsule 210. Lens capsule 210 includes anterior lens capsule portion 212 and posterior lens capsule portion 214. Muscles 208 of the patient are used to reshape the lens capsule 210 and thereby change the shape of the natural lens 202 for use in focusing the natural lens 202. As discussed above, as the natural lens ages, force may be exerting on the trabecular meshwork and canal of schlemm 206 will thereby prevent drainage of aqueous humor from the anterior chamber 220 of the patients eye 200.

The upper half of FIG. 6A shows the eye 200 including one configuration of the present invention in which an intraocular insert has been placed into the lens capsule 210 following extraction of the natural lens 202. In this example, the intraocular insert is circular substantially flat or planar and comprises an artificial lens 230. The lens 230 includes haptics 232 which extend substantially in the plane of the lens and operate the center the lens 230 in the lens capsule 210. Further, the haptics 232 cause the outer radial edge 239 of the lens capsule 210 to expand radially. Note that in the configuration in the upper half of FIG. 6A, the anterior lens capsule portion 212 has been moved away from the cornea 204 relative to the position of the anterior portion 212 with the natural lens 202 in place (lower portion of FIG. 6A). Further, zonules 240 (which secure the lens capsule 210 to the ciliary body muscles 208) are repositioned at an angle relative to the cornea 204 which is reduced with respect to the angle formed by zonules 240 when the natural lens 202 is in place.

In the upper portion of FIG. 6B, another configuration is shown in which the lens haptics 232 have an angle of about 10° and the artificial lens 230 is substantially planar. Again, in this configuration, the canal of schlemm and trabecular meshwork are encouraged to open due to the reshaping of the lens capsule 210. In the example shown in FIG. 6C, the artificial lens 230 has a posterior vault and the haptics 232 have a 10° angle. Again, the re-shaped lens capsule 210 operates to allow the canal of schlemm and trabecular meshwork 206 to open thereby promotes drainage of the aqueous humor.

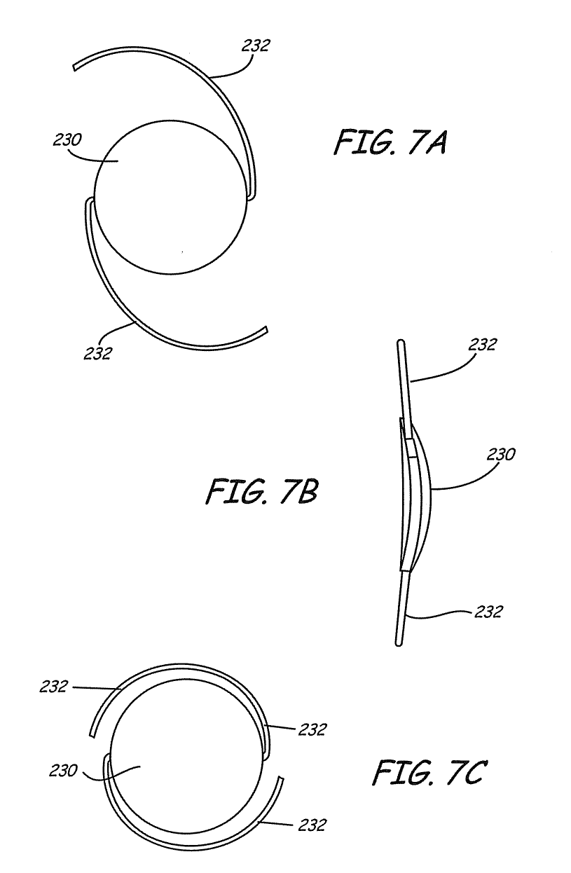

FIG. 7A is a front plan view and FIG. 7B is a side plan view of artificial lens 230 including haptics 232. The body of lens 230 and haptics 232 are formed of deformable materials. In FIGS. 7A and 7B, the haptics 232 of lens 230, along with the lens body, are shown in an unenergized or relaxed state. However, during insertion into the lens capsule 210, the haptics 232 and body of lens 230 are deformed so that they may fit within the lens capsule. Once the lens 230 is inserted into the lens capsule 210, the haptics 232 and body of the lens 230 expand and fill the capsule 210. However, the diameter of the capsule 210 is somewhat smaller than the diameter of the haptics 232 in the relaxed state. Once the haptics 232 expand within the lens capsule 210, the haptics 232 press against the outer periphery 239 of the lens capsule 210. Thus, when the lens 230 is inserted into the lens capsule 210, the lens 230 remains in an energized state and thereby re-shapes the lens capsule 210 into a desired configuration such as those shown in FIGS. 6A-C.

The haptics, or other components of the intraocular insert, can be configured to have the desired elasticity and shape retention characteristics as desired. These can be through the particular materials selected and/or the configuration and shape of the materials. In FIG. 7C, the haptics are shown in a semi-energized condition in which they extend to the outer periphery 239 of the lens capsule 214. This forms the lens capsule 210 into a desired shape.

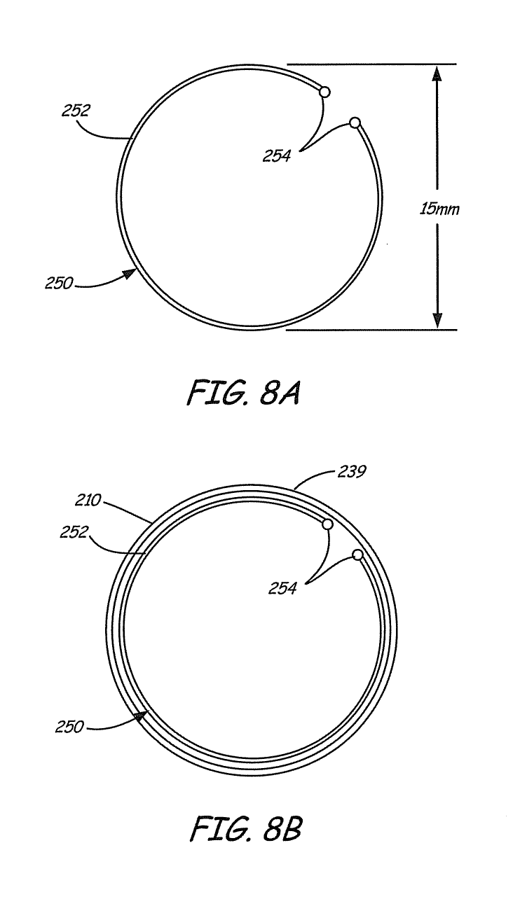

FIGS. 8A and 8B show another example of an intraocular insert in accordance with the present invention. In FIGS. 5A and 8B a capsular tension ring 250 is shown. In FIG. 8A, the ring 250 is shown in a relaxed state. As discussed above with respect to lens 230, the ring 250 is formed of a deformable elastic material. The tension ring 250 includes midsection 252 which is bounded by ends 254. The tension ring 250 is inserted through an incision in the cornea 205. It is moved through the opening in the anterior capsule 215 and into the lens capsular sack 210. This causes deformation of the ring 250 into an energized state in which the material of the ring 250 attempts to return to its unenergized or relaxed condition as shown in FIG. 8A. Once the ring 250 is in the capsular sack 210, it expands and pushes against the outer periphery 239 of the lens capsule 210 as shown in FIG. 8B. In the configuration shown in FIG. 8B, the diameter of the ring 250 is less than the diameter shown in FIG. 8A of ring 250 when the ring 250 is in a relaxed condition. This urges the lens capsule 210 into a desired shape as discussed above with respect to FIGS. 6A, 6B and 6C.

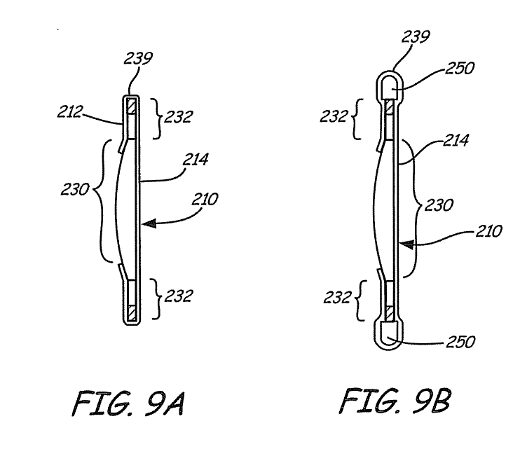

FIGS. 9A and 9B are cross-sectional views which illustrate another example configuration of the present invention in which two intraocular inserts are employed to deform the lens capsule 210 as desired. In cross-section view of FIG. 9A, a lens 230 is shown in the capsular sac 210 having haptics 232. This configuration operates as discussed above in connection with FIG. 6A. The cross-sectional view of FIG. 9B shows another embodiment using a second intraocular insert. In this example, a capsular tension ring 250 is used along with the intraocular lens 230. The combination of two intraocular inserts can provide additional control over the re-shaping of the capsular sac 210. For example, in the example shown in FIGS. 9A and 9B, the outer periphery 239 of the capsular sac 210 is extended through the use of a second intraocular insert.

The present invention includes a method and apparatus for reducing intraocular pressure. In one example configuration, the present invention includes placing a deformable intraocular insert into a patient's eye. The intraocular insert has elastic properties and, prior to insertion, is in a relaxed state. During the insertion process, the insert experiences a first energized state. Following insertion, the insert changes shape into a second energized state in which the capsular sac (lens capsule) is reshaped and/or repositioned as desired.

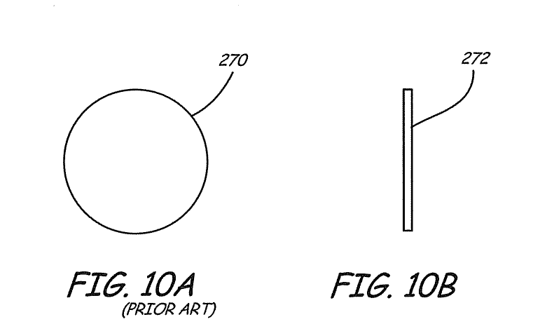

The intraocular insert can be configured to reshape the capsular sac as desired. This can be achieved, for example, through the use of particular materials, and/or configurations of the insert. For example, FIG. 10A is a cross-sectional view of a prior art haptic 270. Haptic 270 has a generally circular cross-section. In FIG. 10B, a cross-sectional view of a haptic 272 is shown which has a rectangular profile. In this configuration, the rectangular profile will have an increased stiffness along its thicker profile relative to the thinner profile. For example, this arrangement can be used to increase the diameter of the insert subsequent to implantation, or increase the force against the outer periphery 239 of the lens capsule.

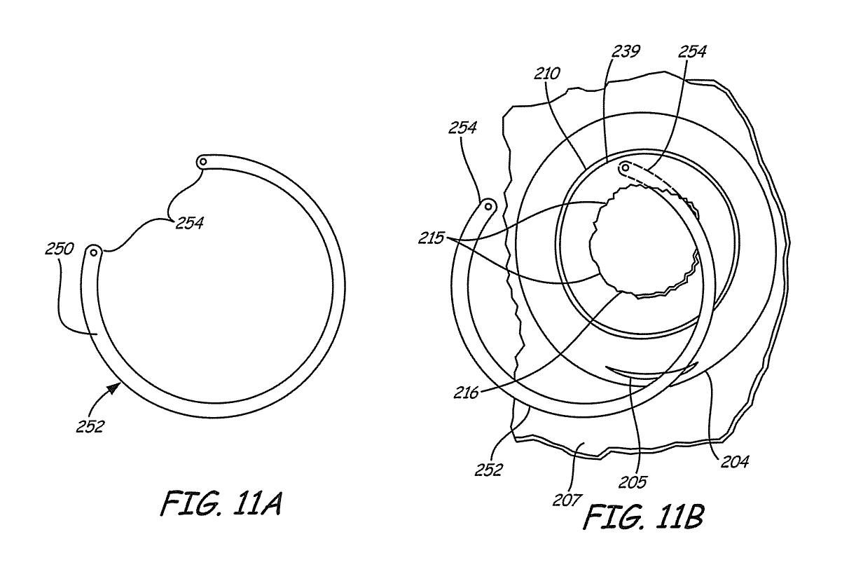

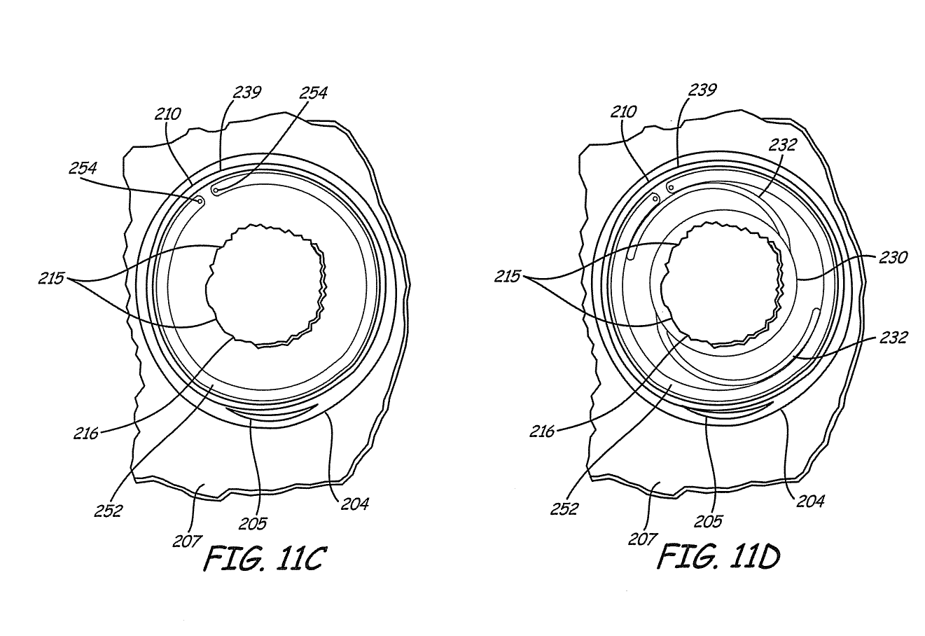

FIG. 11A is another plan view of capsular tension ring 252 in the relaxed state. In FIG. 11B, the capsular tension ring 252 is illustrated in another energized state during the implantation process. In this process, the capsular tension ring 252 is deformed as it is urged through corneal incision 205. This provides a second energized state. A first energized state is illustrated in FIG. 11C following the implantation of the capsular tension ring 252. FIG. 11D shows the capsular tension ring 252 implanted along with an intraocular lens body 230 having haptics 232.

With the present invention, an intraocular member is provided which, in an energized state, is configured to urge deformation of the lens capsule. This deformation can be a stretching or other alteration of the lens capsule to reconfigure the shape of the capsule as desired. For example, the shape of the anterior capsular rim can be made a planar straight line from an edge of the capsular hexis opening (shown in Figures) to the apex of the capsule, with the shape of the posterior capsular wall curvilinear such that the locations of the zonular attachments to the lens capsule are oriented more rearward to thereby exert an increased rearward tension on the ciliary body.

Although the present invention has been described with reference to preferred embodiments, workers skilled in the art will recognize that changes may be made in form and detail without departing from the spirit and scope of the invention. The particular replacement lens can be chosen as desired. In general, there are two basic lens types, a plate lens which is one solid piece with an ovoid or round in shape. A second type is a haptic lens with either an ovoid or a round body which has two or more curved, spring-like plastic arms which extend from the body of the lens. One example plate lens is the Staarr lens available from Staar Surgical Company of Monrovia, Calif. Example haptic lenses are SI-30NB and the SI-40NB available from Advanced Medical Optics, Inc. (AMO) of Santa Ana, Calif.

What is claimed is:

1. An apparatus for reducing intraocular pressure in a patient's eye, comprising:
a deformable intraocular member having:
a relaxed state with a relaxed profile having a first width;
a first energized state with a first profile having a second width;
a second energized state with second profile having a third width;
wherein the deformable intraocular member relaxed state width is greater than the width of a lens capsule which surrounds a lens of the patient's eye after cataract surgery, the first energized state width is arranged for implantation of the deformable intraocular member into the lens capsule through the circular opening in the anterior lens capsule, and the third energized state width is greater than the patient's lens capsule width after cataract surgery and smaller than the width in the relaxed state;
wherein the deformable intraocular member in the second energized state is configured to urge deformation of the lens capsule to thereby promote opening a trabecular meshwork and canal of Schlemm of the patient's eye to promote drainage of aqueous humor from the anterior chamber and reduce intraocular pressure in the patient's eye.

2. The apparatus of claim 1 wherein the deformable intraocular member comprises a lens.

3. The apparatus of claim 1 wherein the lens includes a rearward vault.

4. The apparatus of claim 2 wherein a cross-section of the at least one haptic is non-circular.

5. The apparatus of claim 2 wherein the cross-section of the haptic is rectangular.

6. The apparatus of claim 2 wherein the vault has an angle of about between 1° and 20°.

7. The apparatus of claim 2 wherein the lens includes at least one haptic.

8. The apparatus of claim 7 wherein the vault has an angle of about 3° to about 10°.

9. The apparatus of claim 2 wherein the lens has an A-constant which is greater than about 118.0.

10. The apparatus of claim 2 wherein the A-constant is greater than about 118.5.

11. The apparatus of claim 1 wherein the deformation of the deformable intraocular member capsule causes a force to be exerted on zonules of the patient's eye.

12. The apparatus of claim 11 wherein the force has a component which is directed toward a posterior of the patient's eye.

13. The apparatus of claim 1 wherein the deformable intraocular member comprises a capsular tension ring.

14. The apparatus of claim 13 wherein a cross-section of the capsular tension ring is non-circular and it is made of material enabling a greater expansion force.

14. The apparatus of claim 13 wherein a cross-section of the capsular tension ring is non-circular.

15. The apparatus of claim 13 wherein a cross-section of the tension ring is circular and is formed of a material having increased resistance to compression to thereby increase its expansion ability and provide increased expansion when compression is released.

16. The apparatus of claim 1 wherein the deformable intraocular member comprises an artificial lens and a capsular tension ring.

17. The apparatus of claim 15 wherein the artificial lens includes at least one haptic.

18. The apparatus of claim 16 wherein the artificial lens includes at least one haptic.

19. The apparatus of claim 1 wherein the deformable intraocular member comprises an artificial lens which includes at least one haptic, wherein the haptic extends radially in a plane of the lens.

20. The apparatus of claim 1 where the deformable intraocular member comprises an artificial lens which includes at least one haptic, wherein the haptic extends at an angle relative to a plane of the lens.

21. The apparatus of claim 1 wherein the deformable member is arranged to press against an outer periphery of the lens capsule when in the second energized state.
